(19) United States
(12) Patent Application Publication
(10) Pub. No.: US 2006/0292686 A1
(43) Pub. Date: Dec. 28, 2006

(54) Analyte recovering device, and analyte recovering method

(75) Inventor: Tatsuo Fujikura, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 11/475,072

(22) Filed: Jun. 27, 2006

(30) Foreign Application Priority Data
Jun. 27, 2005 (JP) 2005-187202

(51) Int. Cl. C12M 1/34

(52) U.S. Cl. 435/287.2

(57) ABSTRACT

An analyte recovering device which supplies an analyte solution containing an analyte to a flow path including a substantially flat face on which a ligand is attached, for binding the ligand and the analyte in the analyte solution with each other, and recovering the bound analyte is provided. The device includes an analyte supply/recovery section; a measuring section which measures a bound state between the ligand and the analyte, and a determination section which determines whether the binding is in a saturated state. When it is determined by the determination section that the binding is not in the saturated state, the analyte solution recovered by the analyte supply/recovery section is supplied back to the flow path, and the measurement and the determination are performed, and when it is determined that the binding is in the saturated state, the recovery of the analyte bound to the ligand is performed.

### Cross-Reference to Related Application

This application claims priority under 35 USC 119 from Japanese Patent Application No. 2005-187202, the disclosure of which is incorporated by reference herein.

### BACKGROUND OF THE INVENTION

1. Field of the Invention

The present invention relates to an analyte recovering device and an analyte recovering method, which supplies an analyte solution containing an analyte to a ligand to bind the ligand and the analyte with each other, and recover the analyte bound to the ligand.

2. Description of the Related Art

Conventionally, a process in which, to an attached ligand, an analyte which interacts with this ligand is bound; thereafter, the bound analyte is dissociated from the ligand for recovery; and the recovered analyte is subjected to various analyses has been carried out (referring to, for example, Japanese Unexamined Patent Publication No. 9-500208, Japanese Unexamined Patent Publication No. 11-512518).

For example, in Japanese Unexamined Patent Publication No. 9-500208, a device which binds an analyte (a ligand-binding substance) to an attached ligand; thereafter, dissociates the analyte with a dissociation liquid; and catches the dissociated analyte with a container for recovery is disclosed. In this device, a flow path is formed on the attached ligand with a flow path member, and into this flow path, a solution containing the analyte is continuously supplied, thereby the analyte is supplied to the attached ligand.

In order to cause the binding of the ligand and the analyte to reach a saturated state, a certain degree of period of time is required. Thus with the method as given in Japanese Unexamined Patent Publication No. 9-500208, it is required to continuously supply the analyte solution for a predetermined period of time. Since the fed analyte solution is discarded after having passed through the flow path, a large amount of analyte solution had been required. In the discarded analyte solution, the analyte which has not been bound to the ligand is left, resulting in the analyte solution being wastefully discarded.

### SUMMARY OF THE INVENTION

The present invention is made in view of the above-mentioned fact, and provides an apalyte recovering method which uses a predetermined amount of analyte solution for efficiently binding the analyte to the ligand, and recovers the bound analyte.

A first aspect of the present invention provides an analyte recovering device which supplies an analyte solution containing an analyte to a flow path including a substantially flat face on which a ligand is attached, for binding the ligand and the analyte in the analyte solution with each other, and recovering the bound analyte, the device including: an analyte supply/recovery section which supplies a predetermined amount of the analyte solution to the flow path, and recovers the supplied analyte solution, a measuring section which measures the bound state between the ligand and the analyte in the analyte solution, and a determination section which, on the basis of the measurement result by the measuring section, determines whether the binding of the ligand and the analyte in the analyte solution is in a saturated state, wherein, when it is determined by the determination section that the binding of the ligand and the analyte in the analyte solution is not in the saturated state, the analyte solution recovered by the analyte supply/recovery section is supplied back to the flow path, and the measurement by the measuring section and the determination by the determination section are performed, and when it is determined by the determination section that the binding of the ligand and the analyte in the analyte solution is in the saturated state, the recovering of the analyte bound with the ligand is performed.

With the analyte recovering device of the above configuration, the analyte solution supplied to the flow path by the analyte supply/recovery section can be recovered. Then, the bound state between the analyte in the supplied analyte solution and the attached ligand is measured by the measuring section, and whether the binding is in the saturated state is determined by the determination section. When the binding of the ligand and the analyte is not in the saturated state, the analyte solution recovered by the analyte supply/recovery section is supplied back to the flow path, and the measurement by the measuring section and the determination by the determination section are carried out again. When it is determined that the binding of the ligand and the analyte is in the saturated state, the recovery of the analyte bound to the ligand is performed.

With the present invention, the analyte solution once supplied is recovered, and supplied back to the flow path for binding with the ligand. Therefore, the analyte solution will not be wastefully discarded, and the analyte can be efficiently bound to the ligand with a small amount of the analyte solution. In addition, the bound state between the ligand and the analyte is measured by the measuring section; from the measurement result, it is determined whether the binding of both is in the saturated state; and when it is in the saturated state, the process proceeds to the subsequent analyte recovering. Therefore, supply of the analyte solution is not wastefully repeated.

Herein, the ligand refers to a high polymer having a physiological activity, and examples thereof include protein, DNA, RNA, saccharide, and the like, but it is not limited to these.

Further, the analyte refers to any kind of compound which is supplied to the ligand in order to test whether it interacts with the ligand.

A pipette, an injection tube connected to the supply pump, or the like, can be used as the analyte supply/recovery section.

The analyte recovering device of the first aspect of the present invention may be configured such that the substantially flat face is configured with a metal film, and the measuring section measures the bound state between the ligand and the analyte by utilizing the total reflection attenuation which is generated by irradiating a light beam to a face of the metal film that is on the reverse side to the side on which the flow path is formed.

Thus, the total reflection attenuation can be utilized for measuring the bound state between the ligand and the analyte.

The analyte recovering device of the first aspect may be further including a dielectric block including a light reflection face, wherein the metal film is formed on the light reflection face of the dielectric block, and the measuring section irradiates a light beam to the metal film through the dielectric block, and causes the irradiated light beam to be reflected from the metal film.

According to the above configuration, the metal film is directly formed on the dielectric block. Therefore, the optical loss can be reduced as compared to a case when the metal film and the dielectric block are independently provided. In addition, when the metal film and the dielectric block are provided independently, refractive index matching oil, or the like, is required to be injected between the dielectric block and the plate on which the metal film is formed. However, with the above-mentioned configuration, there is no need for injecting refractive index matching oil, or the like, thereby the configuration of the recovery device can be simplified, and handling can be easy, resulting in enhanced benefit and convenience.

The analyte recovering device of the first aspect of the present invention may further including: a substantially transparent flat plate; and an optical prism, wherein the metal film is formed on one face of the substantially transparent flat plate, the optical prism is adhered to the face of the flat plate on the reverse side to the side on which the metal film is formed, and the measuring section irradiates a light beam to the metal film through the optical prism, and causes the irradiated light beam to be reflected from the metal film.

According to the above configuration, the flat plate on which the metal film is formed can be provided as an element independent of the dielectric block, which renders the configuration of the device simple.

A second aspect of the present invention provides an analyte recovery method including processes of supplying an analyte solution containing an analyte to a flow path including a substantially flat face on which a ligand is attached, and measuring the interaction between the ligand and the analyte in the analyte solution, the method including: supplying a predetermined amount of the analyte solution to the flow path, measuring a bound state between the ligand and the analyte in the analyte solution, determining, on the basis of the result of the measurement, whether the binding of the ligand and the analyte in the analyte solution is in a saturated state, and recovering the analyte solution supplied to the flow path, wherein, when it is determined that the binding of the ligand and the analyte in the analyte solution is not in the saturated state, the analyte supplying, the measuring, and the analyte solution recovering are repeated, and when it is determined that the binding of the ligand and the analyte in the analyte solution is in the saturated state, the analyte bound with the ligand is recovered.

With the above-mentioned analyte recovering method, a predetermined amount of analyte solution is supplied to the flow path for measuring a bound state between the ligand and the analyte in the analyte solution. On the basis of the measurement result, it is determined whether the binding of the ligand and the analyte in the analyte solution is in a saturated state. When it has been determined that the binding of the ligand and the analyte in the analyte solution is not in the saturated state, the analyte supplying, the measurement, and the analyte solution recovering are repeated. When it has been determined that the binding of the ligand and the analyte in the analyte solution is in a saturated state, the analyte bound to the ligand is recovered.

According to the present invention, the analyte solution once supplied is recovered, and supplied back to the flow path for binding with the ligand. Therefore, the analyte solution will not be wastefully discarded, and the analyte can be efficiently bound to the ligand with a small amount of the analyte solution. In addition, the bound state between the ligand and the analyte is measured; from the measurement result, it is determined whether the binding of both is in a saturated state; and when it is in a saturated state, the analyte is recovered. Therefore, supply of the analyte solution is not wastefully repeated.

The analyte recovering method of the second aspect of the present invention may be such that recovery of the analyte bound with the ligand is carried out by supplying a dissociation solution to the flow path for dissociating the ligand and the analyte, and recovering the supplied dissociation solution.

Thus, by using the dissociation liquid for dissociating the analyte from the ligand to dissolve the analyte into the dissociation liquid, the analyte dissociated from the ligand can be recovered together with the dissociation liquid.

The analyte recovering method of the second aspect may be configured such that the substantially flat face is configured with a metal film, and the measurement comprises measuring the bound state between the ligand and the analyte by utilizing the total reflection attenuation which is generated by irradiating a light beam to the face of the metal film that is on the reverse side to the side on which the flow path is formed.

Thus, the total reflection attenuation can be utilized for measuring the bound state between the ligand and the analyte.

The analyte recovering method of the second aspect may be such that the metal film is formed on a light reflection face of a dielectric block, and the measurement comprises irradiating a light beam to the metal film through the dielectric block, and causing the irradiated light beam to be reflected from the metal film.

According to the above configuration, the metal film is directly formed on the dielectric block. Therefore, the optical loss can be reduced as compared to that when the metal film and the dielectric block are provided independently. In addition, there is no need for using refractive index matching oil, or the like, thereby the configuration of the recovery device can be simplified, and handling can be easy, resulting in enhanced benefit and convenience.

The analyte recovering method of the second aspect may be such that the metal film is formed on one face of a substantially transparent flat plate, and the measurement comprises irradiating a light beam to the metal film through an optical prism which is adhered to a face of the flat plate on the reverse side to the side on which the metal film is formed, and causing the irradiated light beam to be reflected from the metal film.

According to the above configuration, the flat plate on which the metal film is formed can be provided as an element independent of the dielectric block, which renders the configuration of the device simple.

Because the present invention provides the above configuration, the analyte can be efficiently bound to the ligand with a predetermined amount of analyte solution, and then the bound analyte can be recovered.

### BRIEF DESCRIPTION OF THE DRAWINGS

An embodiment of the present invention will be described in detail based on the following figures, wherein:

FIG. 1 is a general perspective view of a biosensor of the present embodiment;

FIG. 2 is a perspective view of a sensor stick of the present embodiment;

FIG. 3 is an exploded perspective view of the sensor stick of the present embodiment;

FIG. 4 is a sectional view of the liquid flow path portion of the sensor stick of the present embodiment;

FIG. 5 is a drawing illustrating a state in which a light beam is irradiated to the measurement region and the reference region of the sensor stick of the present embodiment, respectively;

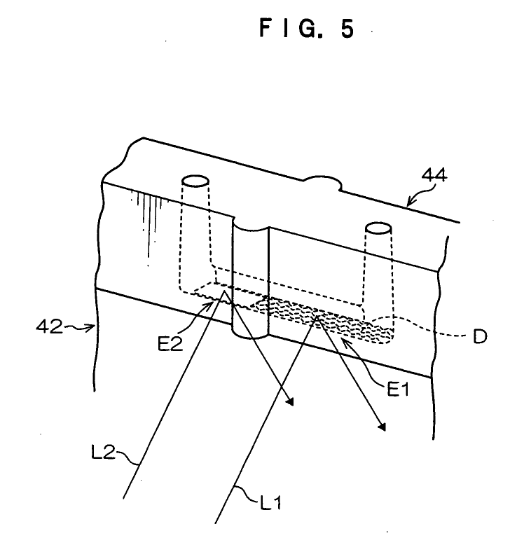

FIG. 6A to FIG. 6C are side views of the pipette part constituting the liquid supply section of the present embodiment;

FIG. 7 is a schematic drawing for the area around an optical measuring section of the biosensor of the present embodiment;

FIG. 8 is a schematic block diagram of a control section and the related section of the present embodiment;

FIG. 9 is a flowchart for a measurement processing of the present embodiment;

FIG. 10 is a graph illustrating one example of measurement result of the present embodiment;

FIG. 11 is a flowchart for an analyte supply/recovery processing of the present embodiment;

FIG. 12A and FIG. 12B are drawings illustrating a liquid supply operation in the liquid flow path of the present embodiment;

FIG. 13A and FIG. 13B are drawings illustrating a liquid discharge procedure in the liquid flow path of the present embodiment; and

FIG. 14 is a schematic sectional view of a sensor chip of another embodiment:

### DETAILED DESCRIPTION OF THE INVENTION

The analyte recovering device of the present invention is configured as a biosensor 10. The biosensor 10 is a so-called surface plasmon sensor which utilizes the surface plasmon resonance occurring at the surface of a metal film for measuring an interaction between a ligand D and an analyte A.

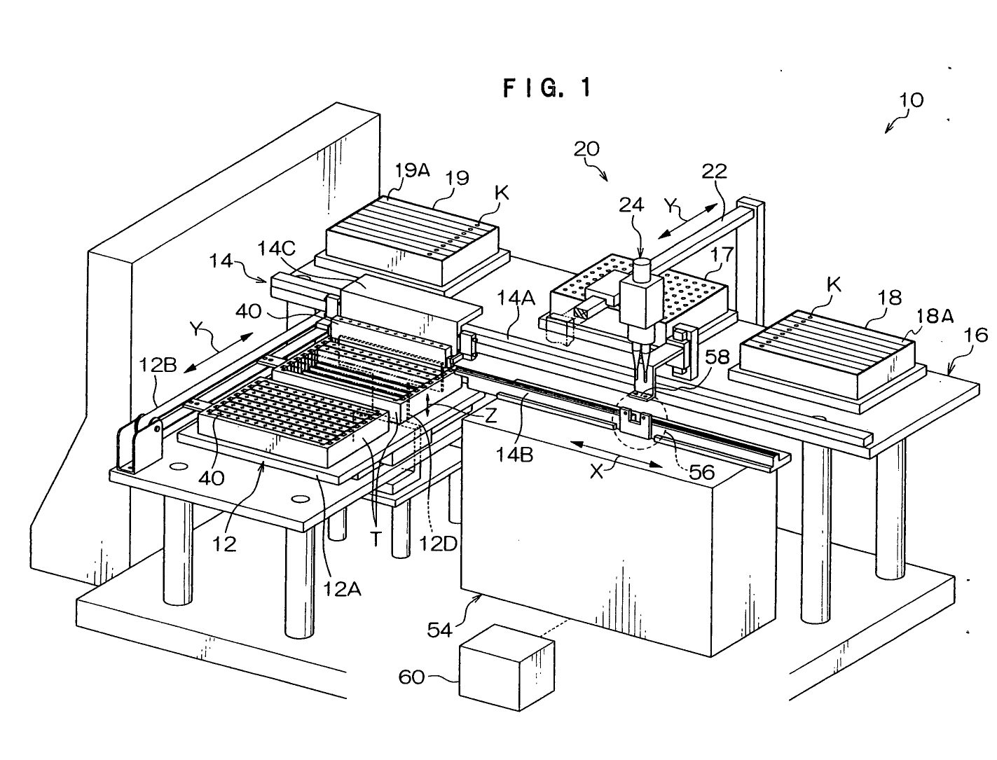

As shown in FIG. 1, the biosensor 10 includes a tray holding member 12, a transferring member 14, a container table 16, a liquid supply/discharge section 20, a measuring section 56, an optical measuring section 54, and a control section 60.

The tray holding member 12 is configured to include a mounting table 12A, and a belt 12B. The mounting table 12A is mounted to the belt 12B extending in a direction of arrow Y, and can be moved in the direction of arrow Y by rotation of the belt 12B. On the mounting table 12A, two trays T are placed and positioned. For example, the tray T accommodates eight sensor sticks 40. The sensor stick 40 provides a chip on which the ligand D is attached, and will be described later in detail. Under the mounting table 12A, a pushing-up mechanism 12D is disposed. The pushing-up mechanism 12D pushes up the sensor stick 40 to the position where it is held by a stick holding member 14C later described.

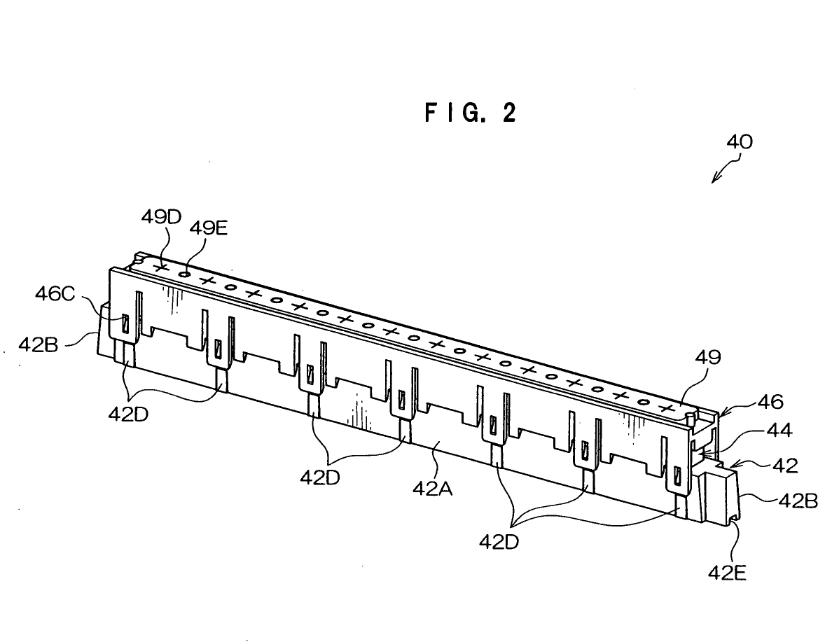

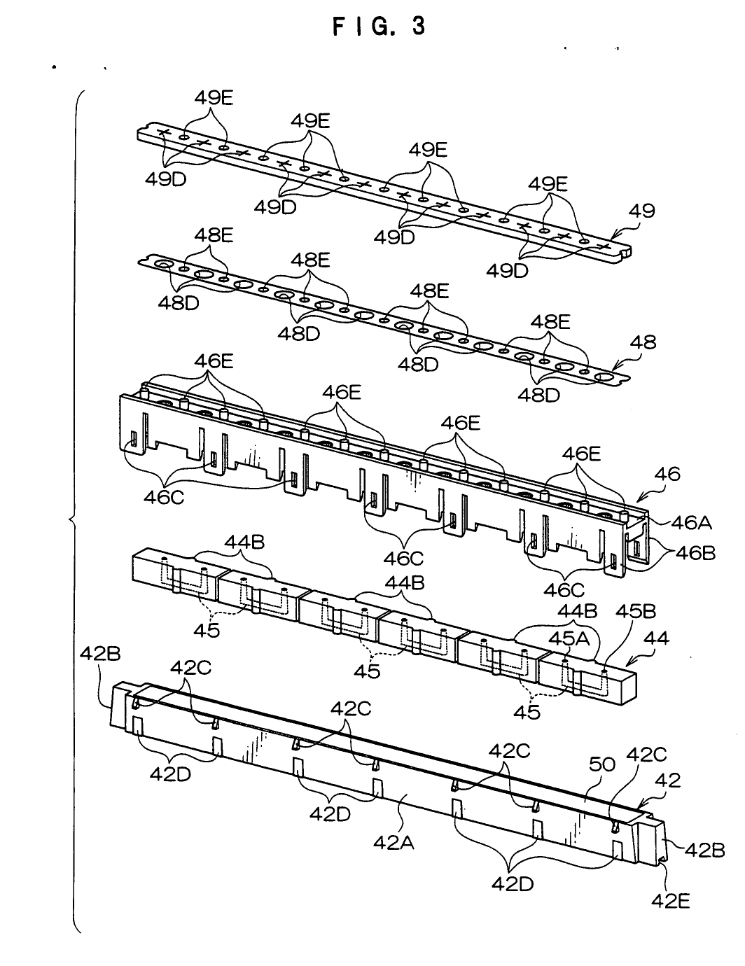

As shown in FIG. 2 and FIG. 3, the sensor stick 40 is configured with a dielectric block 42, a flow path member 44, a holding member 46, an adhesion member 48, and an evaporation prevention member 49.

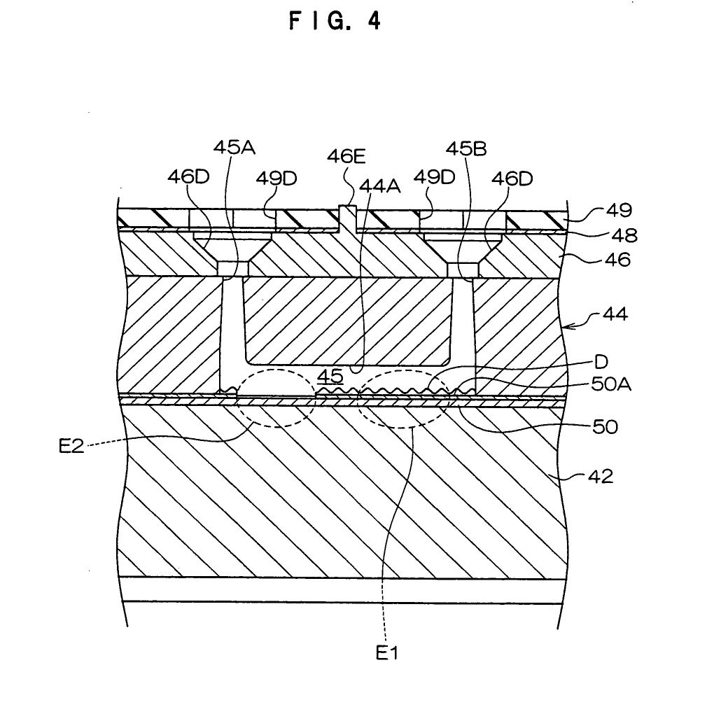

The dielectric block 42 is configured with a substantially transparent resin, or the like, which is substantially transparent to a light beam, and includes a prism portion 42A which is formed in the shape of a bar having a trapezoid section, and a supported (to-be-held) portion 42B at both ends of the prism portion 42A that is formed integrally with the prism portion 42A. As shown also in FIG. 4, a metal film 50 is formed on the top face of the prism portion 42A, which is a wider one of the two faces which is parallel with each other. On this metal film 50, the ligand D which is to be analyzed with the biosensor 10 is attached. The dietelectric, block 42 functions as a kind of prism. In measurement with the biosensor 10, a light beam is irradiated to one of the two opposite side faces of the prism portion 42A that are not parallel with each other, and from the other, the light beam totally reflected at the boundary face of the metal film 50 is emitted.

As shown in FIG. 4, a linker layer 50A is formed on the surface of the metal film 50. The linker layer 50A is a layer for immobilizing the ligand D on the metal film 50. On the linker layer 50A, a measurement region (E1) where the ligand D is attached and reaction between the analyte A and the ligand D occurs, and a reference region (E2) where the ligand D is not attached, and which is for obtaining a reference signal in signal measurement with the measurement region E1 are formed. This reference region E2 is formed in forming a film of the above-mentioned linker layer 50A. A method for forming the reference region E2 is, for example, to subject the linker layer 50A to a surface treatment (blocking) for deactivation of the coupling group which couples to the ligand D. Thereby, a half of the linker layer 50A is provided as the measurement region E1, and the remaining half is as the reference region E2. In order to deactivate the coupling group in this way, ethanolamine hydrochloride can be used. As another method for forming the reference region E2 is to dispose an alkyl thiol, for example, instead of carboxymethyl dextran in a region which is to be the reference region E2. In this way, an alkyl group can be disposed on the surface of the region, and because the alkyl group cannot be ligand-coupled by the amino coupling method, the formed region can be used as the reference region E2.

As shown also in FIG. 4, in the portion of the linker layer 50A that is exposed to the liquid flow path 45, other than the reference region E2, the ligand D is attached. In the reference region E2, the ligand D is not attached. Light beams L2 and L1 are irradiated to the reference region E2 and the measurement region E1 respectively. The reference region E2 is a region provided for compensating (correcting) data obtained from the measurement region E1 where the ligand D is attached.

On both side faces of the prism portion 42A, an engaging convex portion 42C which is engaged with the holding member 46, and a vertical convex portion 42D which is configured on the extension of an imaginary plane perpendicular to the top face of the prism portion 42A are formed in a plurality of places (seven in the present embodiment) along the upper and lower edge of the side face, respectively. In addition, in the central portion of the bottom face of the dielectric block 42 that is along the longitudinal direction thereof, an engaging,groove 42E is formed.

The flow path member 44 is formed as a rectangular in which the width is slightly narrower than the dielectric block 42. As shown in FIG. 3, plural (six in the present embodiment) flow path members 44 are disposed and arranged on the metal film 50 on the dielectric block 42. In the bottom face of the respective flow path members 44, a flow path groove 44A is formed to communicate with a supply port 45A and a discharge port 45B which are formed in the top face, constituting a liquid flow path 45 with the metal film 50. Thus, for one sensor stick 40, plural (six herein) independent liquid flow paths 45 are provided. On the side wall of the flow path member 44, a convex portion 44B to be force-fitted into the concave portion (not shown) in the inside of the holding member 46 for securing close contact with the holding member 46 is formed.

Since it is assumed that, for the liquid flow path 45, a liquid containing protein is supplied, it is preferable that the material for the flow path member 44 have no non-specific adsorptivity for proteins in order to prevent the protein from sticking to the flow path member 44.

The holding member 46 is formed in a continuous (long) length, being composed of a top plate 46A and two side plates 46B. In the side plate 46B, engaging holes 46C which are engaged with the engaging convex portions 42C of the dielectric block 42 are formed. The holding member 46 is mounted to the dielectric block 42, sandwiching the six flow path members 44 therebetween, with the engaging hole 46C being engaged with the engaging convex portion 42C. Thereby, the flow path members 44 are mounted to the dielectric block 42. In the top plate 46A, a tapered pipette insertion hole 46D which is narrowed down toward the flow path member 44 is formed in a position opposed to the supply port 45A and the discharge port 45B of the flow path member 44, respectively. In addition, a positioning boss 46E is formed between the adjacent pipette insertion holes 46D.

The evaporation prevention member 49 is adhered to the top face of the holding member 46 by the adhesion member 48. In the adhesion member 48, a hole 48D for pipette insertion is formed in a position opposed to the pipette insertion hole 46D, and a positioning hole 48E is formed in a position opposed to the boss 46E. In addition, in the evaporation prevention member 49, a slit 49D, which is a cross-shaped cutout, is formed in a position opposed to the pipette insertion hole 46D, and a locating hole 49E is formed in a position opposed to the boss 46E. By inserting the boss 46E into the holes 48E and 49E for adhering the evaporation prevention member 49 to the top face of the holding member 46, the evaporation prevention member 49 is configured such that the slit 49D in the evaporation prevention member 49 is opposed to the supply port 45A and the discharge port 45B of the flow path member 44, respectively. When a pipette tip CP is not inserted, the slit 49D covers the supply port 45A and the discharge port 45B, thereby evaporation of the liquid supplied to the liquid flow path 45 is prevented.

As shown in FIG. 1, the transferring member 14 of the biosensor 10 is configured to include an upper guide rail 14A, a lower guide rail 14B, and a stick holding member 14C. The upper guide rail 14A and the lower guide rail 14B are horizontally disposed in the direction of arrow X that is perpendicular to the direction of arrow Y, above the tray holding member 12 and the optical measuring section 54. The stick holding member 14C is mounted on the upper guide rail 14A. The stick holding member 14C can hold the supported portion 42B at both ends of the sensor stick 40, and move along the upper guide rail 14A. The engaging groove 42E of the sensor stick 40 held by the stick holding member 14C and the lower guide rail 14B are engaged with each other, and the stick holding member 14C is moved in the direction of arrow X, thereby the sensor stick 40 is transferred to the measuring section 56 above the optical measuring section 54. Further, in the measuring section 56, a holding-down member 58 for holding down the sensor stick 40 in measurement is provided. The holding-down member 58 can be moved in the Z direction by a drive mechanism (not shown), and presses the sensor stick 40 disposed in the measuring section 56 from above.

On the container table 16, an analyte solution plate 17, a recovery liquid stock container 18, and a dissociation liquid stock container 19 are placed. The analyte solution plate 17 is partitioned into plural (for example, ninety-six) sections for making it possible to stock various analyte solutions. The recovery liquid stock container 18 is made up of plural recovery containers 18A, and in the recovery container 18A, an opening K for allowing a later described pipette tip CP to be inserted thereinto is formed. The dissociation liquid stock container 19 is made up of plural stock containers 19A, in each of which an opening K for allowing the pipette tip CP to be inserted thereinto is formed in the same manner as in the recovery container 18A.

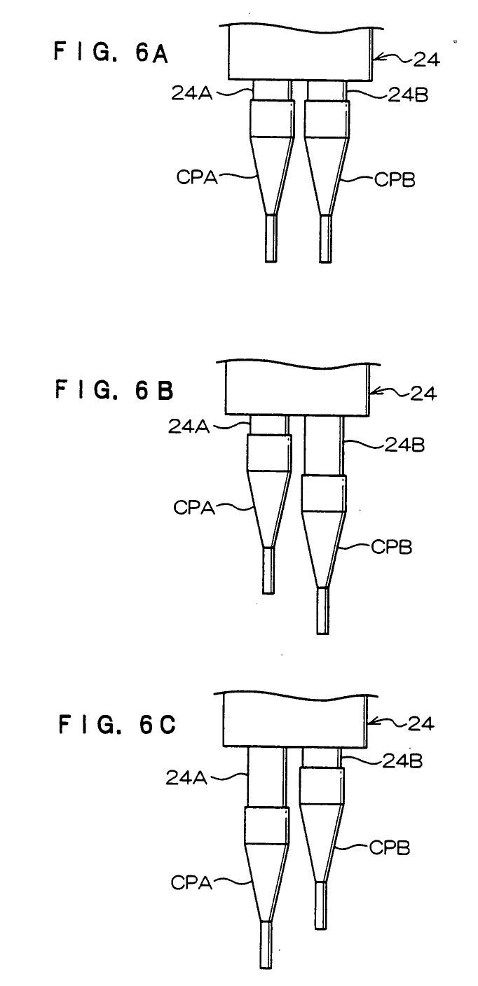

The liquid supply/discharge section 20 is configured to include a traversing rail 22 suspended above the upper guide rail 14A, the lower guide rail 14B in the direction of arrow Y, and a head 24. The traversing rail 22 can be moved in the direction of arrow X by a drive mgchanis,m (not shown). Further, the head 24 is mounted to the traversing rail 22, and can be moved in the direction of arrow Y. The head 24 can be moved also in the vertical direction (in the direction of arrow Z) by a drive mechanism (not shown). As shown in FIG. 6A, the head 24 includes two pipette parts 24A and 24B. The pipette tip CP is mounted at the tip portion to the pipette part 24A and 24B, and the length of the pipette part 24A and 24B in the Z direction can be adjusted respectively. A number of pipette tips CP are stocked in a pipette tip stocker (not shown) so as to allow replacement as needed.

In the present embodiment, liquid supply to the sensor stick 40 is carried out by the pipette tip CP. However, instead of using the pipette tip, for example, an injection tube which one end thereof is connected to the above-mentioned solution plate, and the other can be connected to the sensor stick 40 may be provided and the liquid can be supplied by a supply pump via the injection tube.

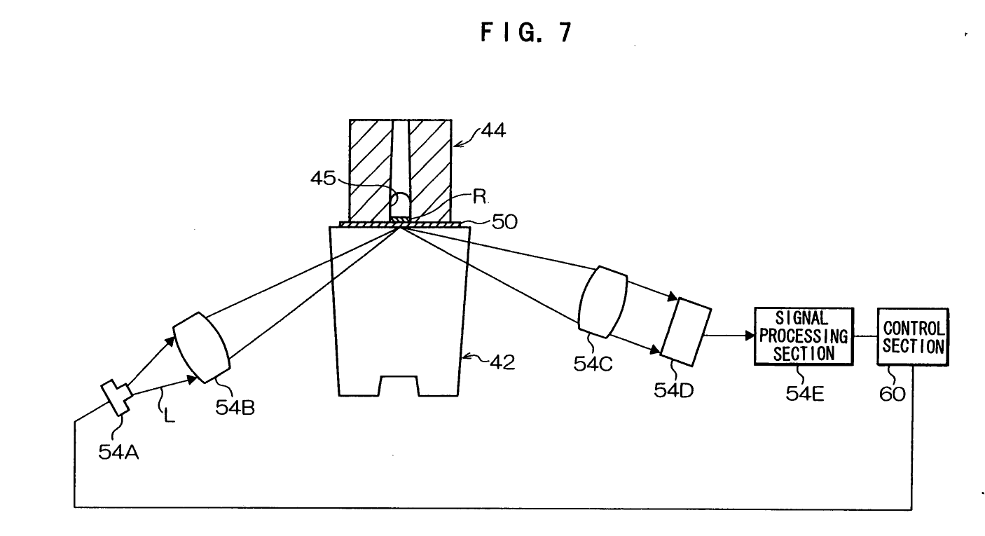

As shown in FIG. 7, the optical measuring section 54 is configured to include a light source 54A, a first optical system 54B, a second optical system 54C, a light receiving section 54D, and a signal processing section 54E. From the light source 54A, a light beam L in diverging state is emitted. The light beam L is changed into two light beams L1 and L2 through the first optical system 54B, being irradiated to the measurement region E1 and the reference region E2 of the dielectric block 42 disposed in the measuring section 56. In the measurement region E1 and the reference region E2, the light beams L1 and L2 are irradiated, including various incident angle components with respect to the boundary between the metal film 50 and the dielectric block 42, and at an angle of the total reflection angle or larger. The light beams L1 and L2 are totally reflected at the boundary between the dielectric block 42 and the metal film 50. The totally reflected light beams L1 and L2 are also reflected with various reflection angle components. These totally reflected light beams L1 and L2 are received by the light receiving section 54D via the second optical system 54C to be photoelectrically converted, respectively, and light detection signals are outputted to the signal processing section 54E. In the signal processing section 54E, a predetermined processing is carried out on the basis of the inputted light detection signals, and data for total reflection attenuation angle (which is hereinafter to be referred as “total reflection attenuation angle data”) for the measurement region E1 and the reference region E2 is determined. This total reflection attenuation angle data is outputted to the control section 60.

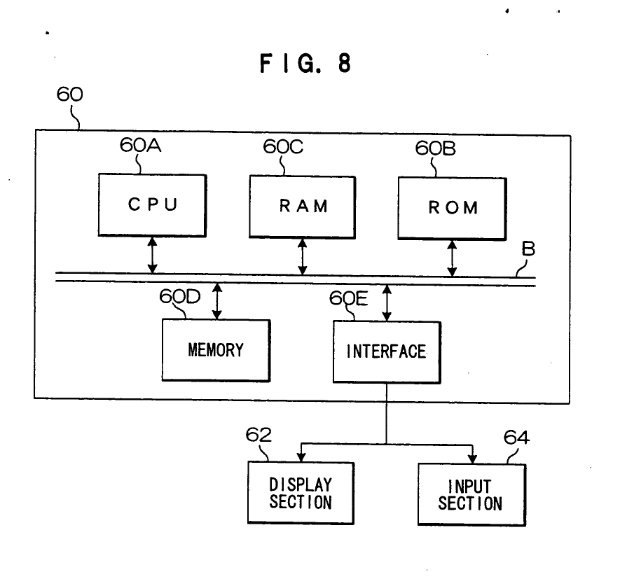

The control section 60 has a function for controlling the entire biosensor 10, and as shown in FIG. 7, is connected to the light source 54A, the signal processing section 54E, and the drive system (not shown) of the biosensor 10. As shown in FIG. 8, the control section 60 includes a CPU 60A, an ROM 60B, an RAM 60C, a memory 60D, and an interface 60E which are mutually connected through a bus B, and is connected to a display section 62 which displays various kinds of information, and an input section 64 for inputting various instructions and various information.

In the memory 60D, various programs and various data for controlling the biosensor 10 are stored.

Next, procedure for recovering the analyte with the biosensor 10 will be described. Herein, the analyte A is bound to the ligand D in the sensor stick 40, and thereafter, only the bound analyte A is dissociated from the ligand D for recovery.

On the mounting table 12A of the biosensor 10, a tray containing the sensor stick 40 in which the ligand D is attached, and which the liquid flow path 45 thereof is filled with a conservation liquid (preservative solution) C is set. Further, in the analyte solution plate 17 and the dissociation liquid stock container 19, a predetermined analyte solution and a supply liquid (a buffer liquid, a dissociation liquid, a cleaning liquid, and the like) are set, respectively.

First, by the pushing-up mechanism 12D, one sensor stick 40 is pushed up to the height level of the stick holding member 14C, and held by the stick holding member 14C. Then, the stick holding member 14C holding the sensor stick 40 is moved along the lower guide rail 14B for transferring the sensor stick 40 to the measuring section 56. The sensor stick 40 transferred to the measuring section 56 is positioned in a predetermined measurement position, and pressed from above by the holding-down member 58 to be fixed.

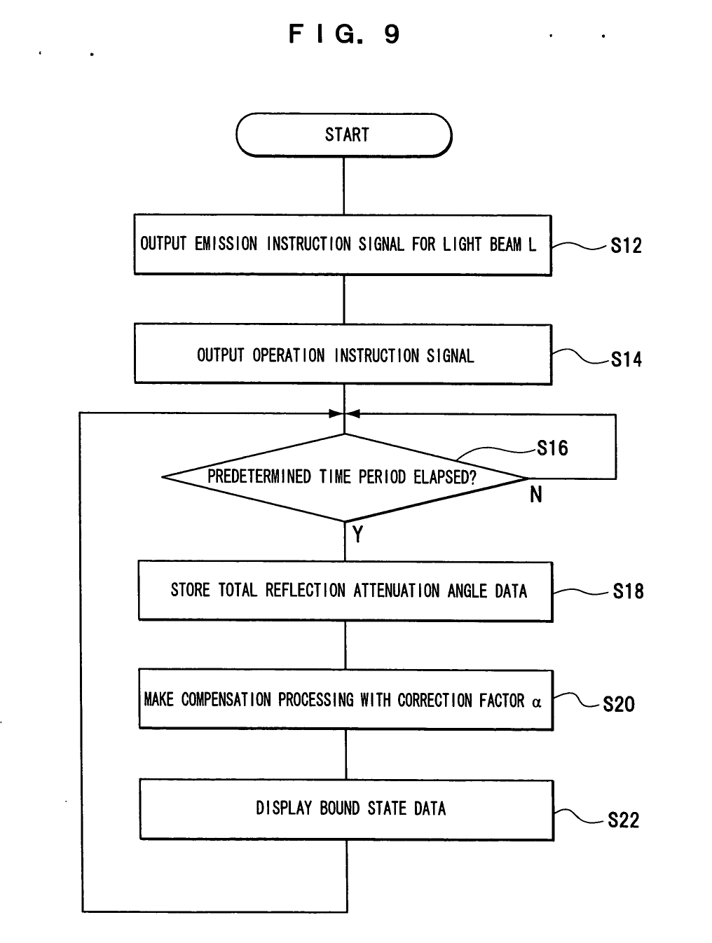

When an instruction for starting the measurement is inputted from the input section 64, the control section 60 performs the measurement processing as shown in FIG. 9.

First, in step S12, an instruction signal for emitting a light beam L is outputted to the light source 54A. Thereby, the light beam L is emitted from the light source 54A. The emitted light beam L is changed into two light beams L1 and L2 by the first optical system 54B, and these are irradiated to the measurement region E1 and the reference region E2 of the liquid flow path 45, respectively. In step S14, an operation instruction signal is outputted to the light receiving section 54D and the signal processing section 54E. Thereby, the light beams L1 and L2 which have been totally reflected by the measurement region E1 and the reference region E2, and passed through the second optical system 54C are received by the light receiving section 54D. The received lights from each of the measurement region E1 and the reference region E2 are photoelectrically converted, and the light detection signals, which are the resultants of the respective conversion, are outputted to the signal processing section 54E. In the signal processing section 54E, the light detection signals are subjected to a predetermined processing, and the total reflection attenuation angle data is generated, respectively, to be outputted to the control section 60.

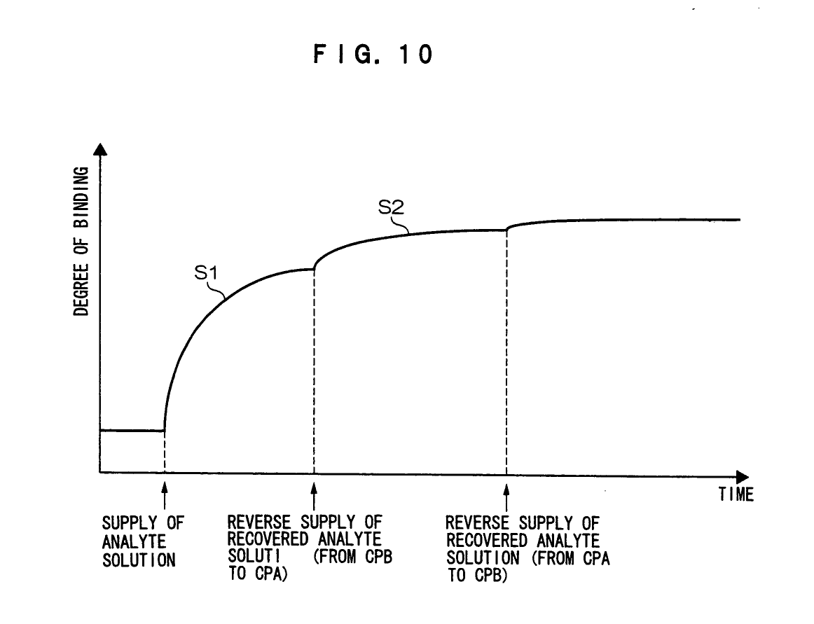

The control section 60 determines whether a predetermined period of time has elapsed in step S16. After a predetermined period of time having elapsed, the inputted total reflection attenuation angle data is stored in the memory 60D at step S18. Then, in step S20, the total reflection attenuation angle data obtained from the light detection signal from the measurement region E1 is compensated (corrected) with the total reflection attenuation angle data obtained from the light detection signal from the reference region E2 for generation of the bound state data indicating the bound state between the ligand D and the analyte A in the analyte solution YA. In step S22, the bound state data is outputted to the display section 62. Thereby, the bound state data for each predetermined period of time is stored in the memory 60D, and displayed by the display section 62. To the display section 62, the bound state data graphed for each predetermined period of time as shown in FIG. 10 is outputted. This measurement processing is continued until a measurement processing completion signal is received.

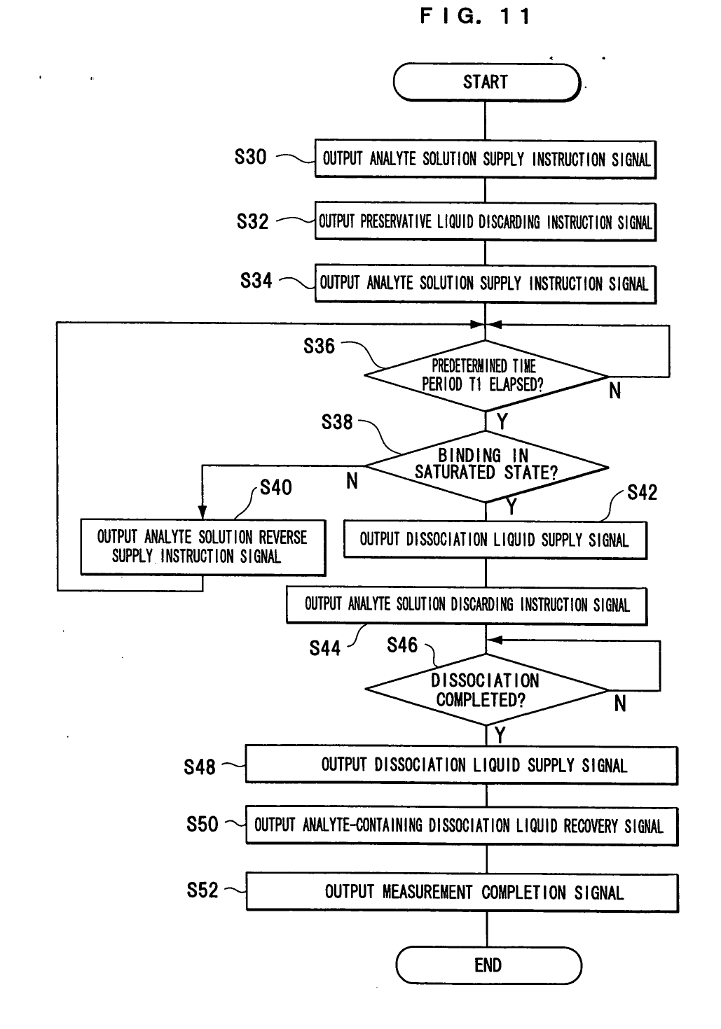

On the other hand, when an instruction for starting the analyte binding recovery process is inputted from the input section 64, the control section 60 implements the analyte binding recovery process as shown in FIG. 11.

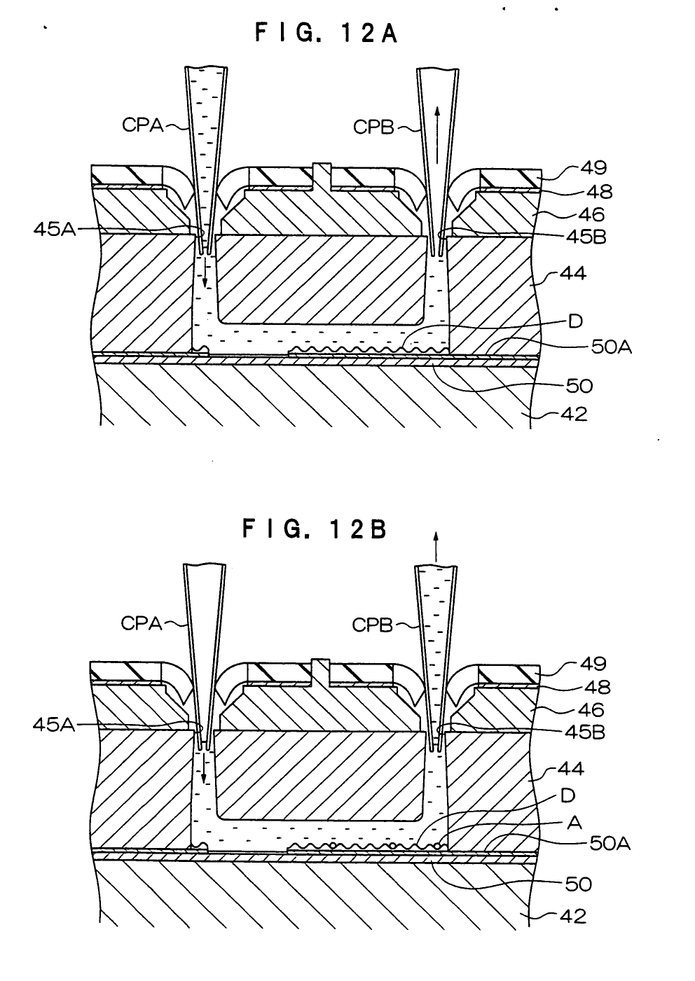

First, in step S30, an instruction signal for supplying the analyte solution YA is outputted. Thereby, the head 24 supplies the analyte solution YA to the liquid flow path 45, and the conservation liquid filled in the liquid flow path 45 is discharged. Supply of the analyte solution YA, and discharge of the conservation liquid are specifically performed in the following manner. First, the head 24 is moved to above the analyte solution plate 17 where the analyte solution YA is set, and between the pipette part 24A and the pipette part 24B, a difference in length along the vertical direction is created such that the former is longer and the latter is shorter (see FIG. 6 (C)). And, the head 24 is lowered to insert only the tip of the pipette tip CPA mounted to the pipette part 24A into the cell in which the analyte solution YA is reservoired, and suck the analyte solution YA into the pipette tip CPA. Next, the head 24 is raised and moved to above the measuring section 56, and the lengths of the pipette part 24A and the pipette part 24B are adjusted such that both are at the same level. Then, the head 24 is lowered to insert the tip of the pipette tip CPA on the pipette part 24A side into the supply port 45A of the liquid flow path 45, and insert the tip of the pipette tip CPB on the pipette part 24B side into the discharge port 45B of the liquid flow path 45 (see FIG. 12A). From the pipette tip CPA to the liquid flow path 45, the analyte solution YA is injected, and with the pipette tip CPB, the conservation liquid forced out from the liquid flow path 45 is sucked (see FIG. 12B). Thereby, the analyte A is supplied to the ligand D, and the conservation liquid is discharged. Further, the ligand D is bound to the analyte A, and to the display section 62, a reaction curve S1, for example, as shown in FIG. 10 is outputted.

At step S32, a conservation liquid discarding instruction signal for directing discarding of the conservation liquid sucked into the pipette tip CPB into the recovery container 18 (18A) is outputted. The discarding herein is performed by moving the head 24 to above the recovery liquid stock container 18; between the pipette part 24B and the pipette part 24A, creating a difference in length along the vertical direction such that the former is longer and the latter is shorter (see FIG. 6 (B)); inserting only the pipette tip CPB mounted to the pipette part 24B into the opening K of the recovery container 18A; and discharging the conservation liquid.

Next, in step S34, an instruction signal for supplying the analyte solution YA is again outputted. Thereby, in this time, the analyte solution YA is supplied to the liquid flow path 45, and the analyte solution YA which has been filled in the liquid flow path 45 is recovered with the pipette tip CPB.

Thereafter, at step S36, the operator awaits for a predetermined period of time, T1, for reaction, and after the predetermined period of time T1 has elapsed, it is determined at step S38 whether the binding of the ligand D and the analyte A is in a saturated state, on the basis of the bound state data obtained by the measurement processing. The determination herein is such that, if the increase in degree of binding in the predetermined period of time T1 from the supply of the analyte solution is equal to or greater than a predetermined rate (for example, 10%), it has been determined that the binding is in progress, and not in a saturated state. If the increase in degree of binding is under the predetermined rate, it can be determined that the binding is in a saturated state.

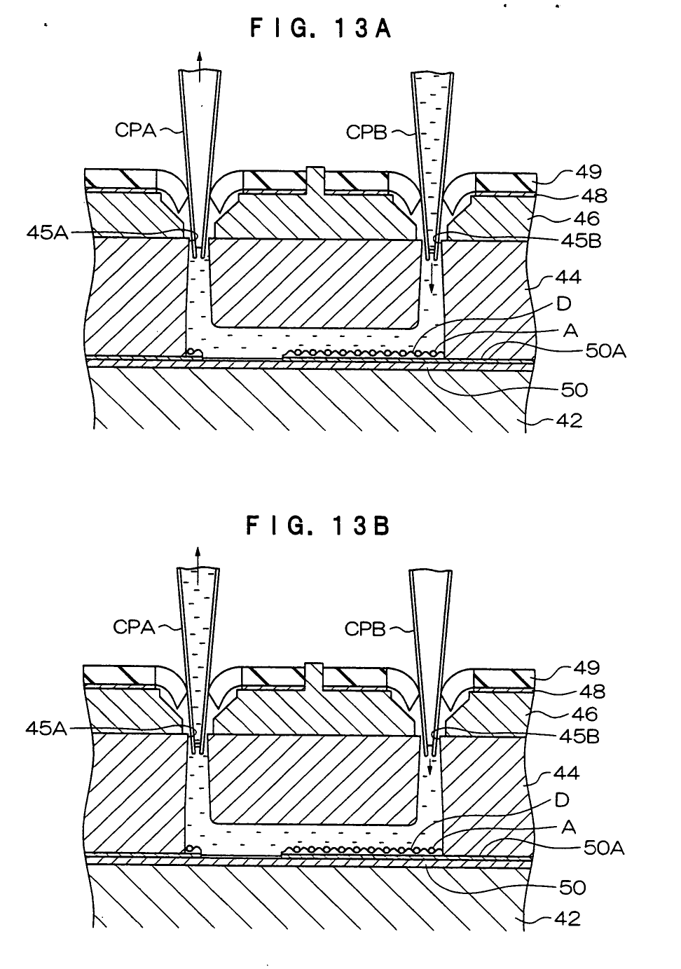

If the determination at step S38 is negative, an analyte solution reverse supply signal is outputted at step S40. As shown in FIG. 13A, the analyte solution YA once recovered into the pipette tip CPB is injected from the discharge port 45B into the liquid flow path 45, and with the pipette tip CPA, the analyte solution YA discharged from the supply port 45A side is recovered. Thereby, the analyte solution YA in the liquid flow path 45 is replaced with the analyte solution YA which has been sucked into the pipette tip CPB. By this replacement operation, the analyte solution YA is stirred, and the non-uniformity in concentration of the analyte A in the liquid flow path 45 being eliminated, thereby the binding to the ligand D being promoted.

After completion of step S40, the process returns to step S36 for repeating the above steps. By such repetition, as shown with the reaction curves SI and S2 in FIG. 10, for example, the degree of binding of the analyte A and the ligand D increases.

When the determination at step S38 is affirmative, the binding of the ligand D and the analyte A is in a saturated state. Therefore, in order to recover the analyte A bound to the ligand D, a dissociation liquid supply signal is outputted in step S42. Thereby, with the pipette tip CPA, the dissociation liquid J is sucked from the dissociation liquid stock container 19 in which the dissociation liquid J is stocked. The sucked dissociation liquid J is supplied to the liquid flow path 45 from the supply port 45A. At this time, the pipette tip CPB is inserted into the discharge port 45B, and the analyte solution YA forced out from the liquid flow path 45 is sucked (see FIG. 13B).

In step S44, an analyte solution discarding instruction signal for directing discarding of the analyte solution YA sucked into the pipette tip CPB into the recovery container 18 is outputted. Thereby, in the same manner as the conservation liquid discarding in step 32, the analyte solution YA is injected into the recovery container 18 (a recovery container 18 different from that in which the conservation liquid is discharged in S32).

In the liquid flow path 45 where the dissociation liquid has been supplied, the analyte A bound to the ligand D is dissociated from the ligand D, and to the display section 62, the reaction curve S2 as shown in FIG. 10, for example, is outputted.

In step S46, on the basis of the bound state data obtained by the measurement processing, it is determined whether or not the dissociation between the ligand D and the analyte A has been completed. The determination herein is such that, if the change rate of the binding is equal to or below a predetermined value (for example 10%), the dissociation can be determined to have been completed. When the determination is negative; the determination in step 46 is repeated.

When the determination is affirmative, a dissociation liquid supply signal is again outputted in step S48 for injecting the dissociation liquid from the supply port 45A, and recovering the dissociation liquid forced out from the discharge port 45B. In the recovered dissociation liquid, the analyte A dissociated from the ligand D is contained (the recovered dissociation liquid is hereinafter to be called the “analyte-containing dissociation liquid”). In step S50, a signal for recovering the analyte-containing dissociation liquid is outputted for injecting the analyte-containing dissociation liquid which has been sucked into the pipette tip CPB, into the recovery container 18 (another recovery container 18A other than the that in which the analyte solution YA is injected in step S44) for recovery.

Thereafter, in step S52, a measurement completion instruction signal is outputted, and the analyte binding recovery process is completed.

On the other hand, the measurement processing is also completed in response to the measurement completion instruction signal being received.

According to the present embodiment, the analyte solution YA which is discharged after being supplied to the liquid flow path 45 is again returned to the liquid flow path 45 for binding of the ligand D and the analyte A, thus can reduce the amount of use of the analyte solution YA.

Moreover, after the analyte solution YA having been once supplied to the liquid flow path 45, the analyte solution YA is further supplied, thus the analyte A in the analyte solution YA is stirred, the variation in concentration of the analyte A being eliminated, and the binding with the ligand D is promoted.

Further, it is determined, from the binding data obtained by the measurement, whether or not the binding of the ligand D and the analyte A is in a saturated state, and when it is determined that the binding is in a saturated state, the analyte A is dissociated from the ligand D and proceed to the recovering process. Therefore, supply of the analyte solution YA will not be wastefully repeated, and thus the processing can be efficiently carried out.

Further, in the present embodiment, recovery of the analyte A bound to the ligand D is performed by supplying the dissociation liquid J to the liquid flow path 45. However, as alternative methods, for example, the flow path member 44 may be removed from the dielectric block 42 for directly supplying the recovery liquid to the analyte A in the bound state for recovery, or the analyte A may be recovered as adhered to the metal film 50 for performing mass spectroscopy.

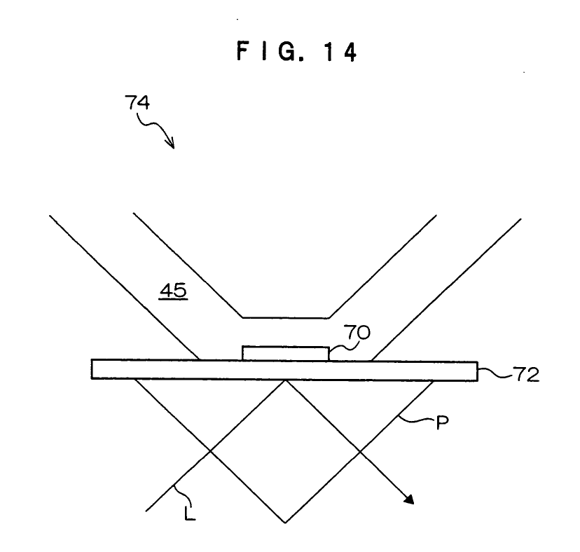

In the present embodiment, the sensor stick 40 in which the metal film 50 where the ligand D is attached is formed on the dielectric block 42 which functions as a prism. However, it is not limited to this. As shown in FIG. 14, a sensor chip 74 in which the metal film 70 is formed on one face of a transparent flat plate 72 may be used. In this case, an optical prism P is tightly adhered to the face of the flat plate 72 on which the metal film 70 is not formed, and through this optical prism P, a light beam L is irradiated to the metal film 70, and the irradiated light beam is reflected therefrom. According to this configuration, the flat plate 72 on which the metal film 70 is formed can be provided as an element independent of the optical prism P, which renders the configuration of the sensor chip 74 simple.

On the other hand, as in the present embodiment, by taking a configuration in which the metal film 50 is formed on the dielectric block 42, the optical loss can be reduced. In addition, when the metal film and the dielectric block are made independent of each other, refractive index matching oil, or the like, is required to be injected between the prism and the plate on which the metal film is formed. However, as in the present embodiment, with the configuration in which the metal film 50 is formed on the dielectric block 42, there is no need for injecting refractive index matching oil, or the like. Thereby, the configuration of the biosensor can be simplified, and handling can be easy, resulting in enhanced benefit and convenience.

In the present embodiment, the surface plasmon sensor is described as one example of the biosensor. However, the biosensor is not limited this. The present invention can be applied to recovery of the analyte using any other biosensors, such as those based on a quartz crystal microbalance (QCM) measurement technology, an optical measurement technology using a functionalized surface ranging from that of gold colloidal particles to that of ultrafine particles, and the like.

Further, as an example of other type of biosensor utilizing the total reflection attenuation, the leakage mode detector can be mentioned. The leakage mode detector is made up of a dielectric, and a thin film constituted by a clad layer and a light guiding layer laminated thereon in this order, one face of this thin film providing a sensor face, and the other face a light incident face. When light is irradiated on the light incident face so as to meet the total reflection conditions, a part thereof permeates the clad layer to be introduced into the light guiding layer. The wave-guiding mode is thereby excited in this light guiding layer, and the reflected light on the light incident face is greatly attenuated. The incident angle at which the wave-guiding mode is excited varies depending upon the refractive index for the medium on the sensor face as with the surface plasmon resonance angle. By detecting the attenuation of this reflected light, the reaction on the sensor face can be measured.

What is claimed is:

1. An analyte recovering device which supplies an analyte solution containing an analyte to a flow path including a substantially flat face on which a ligand is attached, for binding the ligand and the analyte in the analyte solution with each other, and recovering the bound analyte, the device comprising:
an analyte supply/recovery section which supplies a predetermined amount of the analyte solution to the flow path, and recovers the supplied analyte solution,
a measuring section which measures the bound state between the ligand and the analyte in the analyte solution, and
a determination section which, on the basis of the measurement result by the measuring section, determines whether the binding of the ligand and the analyte in the analyte solution is in a saturated state,
wherein, when it is determined by the determination section that the binding of the ligand and the analyte in the analyte solution is not in the saturated state, the analyte solution recovered by the analyte supply/recovery section is supplied back to the flow path, and the measurement by the measuring section and the determination by the determination section are performed, and
when it is determined by the determination section that the binding of the ligand and the analyte in the analyte solution is in the saturated state, the recovering of the analyte bound with the ligand is performed.

2. The analyte recovering device of claim 1, wherein the substantially flat face is configured with a metal film, and
the measuring section measures the bound state between the ligand and the analyte by utilizing the total reflection attenuation which is generated by irradiating a light beam to a face of the metal film that is on the reverse side to the side on which the flow path is formed.

3. The analyte recovering device of claim 2, further comprising a dielectric block including a light reflection face,
wherein the metal film is formed on the light reflection face of the dielectric block, and
the measuring section irradiates a light beam to the metal film through the dielectric block, and causes the irradiated light beam to be reflected from the metal film.

4. The analyte recovering device of claim 2, further comprising:
a substantially transparent flat plate; and
an optical prism,
wherein the metal film is formed on one face of the substantially transparent flat plate,
the optical prism is adhered to the face of the flat plate on the reverse side to the side on which the metal film is formed, and
the measuring section irradiates a light beam to the metal film through the optical prism, and causes the irradiated light beam to be reflected from the metal film.

5. An analyte recovery method including processes of supplying an analyte solution containing an analyte to a flow path including a substantially flat face on which a ligand is attached, and measuring the interaction between the ligand and the analyte in the analyte solution, the method comprising:
supplying a predetermined amount of the analyte solution to the flow path,
measuring a bound state between the ligand and the analyte in the analyte solution,
determining, on the basis of the result of the measurement, whether the binding of the ligand and the analyte in the analyte solution is in a saturated state, and
recovering the analyte solution supplied to the flow path,
wherein, when it is determined that the binding of the ligand and the analyte in the analyte solution is not in the saturated state, the analyte supplying, the measuring, and the analyte solution recovering are repeated, and when it is determined that the binding of the ligand and the analyte in the analyte solution is in the saturated state, the analyte bound with the ligand is recovered.

6. The analyte recovering method of claim 5, wherein recovery of the analyte bound with the ligand is carried out by supplying a dissociation solution to the flow path for dissociating the ligand and the analyte, and recovering the supplied dissociation solution.

7. The analyte recovering method of claim 5, wherein the substantially flat face is configured with a metal film, and
the measurement comprises measuring the bound state between the ligand and the analyte by utilizing the total reflection attenuation which is generated by irradiating a light beam to the face of the metal film that is on the reverse side to the side on which the flow path is formed.

8. The analyte recovering method of claim 7, wherein the metal film is formed on a light reflection face of a dielectric block, and
the measurement comprises irradiating a light beam to the metal film through the dielectric block, and causing the irradiated light beam to be reflected from the metal film.

9. The analyte recovering method of claim 7, wherein the metal film is formed on one face of a substantially transparent flat plate, and
the measurement comprises irradiating a light beam to the metal film through an optical prism which is adhered to a face of the flat plate on the reverse side to the side on which the metal film is formed, and causing the irradiated light beam to be reflected from the metal film.
